(19) United States
(12) Patent Application Publication
(10) Pub. No.: US 2008/0097617 A1
(43) Pub. Date: Apr. 24, 2008

(54) Ankle Joint Endoprosthesis Elements

(75) Inventors: Michael Fellinger, Graz (AT); Ernst Orthner, Wels (AT); Robert Siorpaes, St. Johann (AT)

Correspondence Address: WORKMAN NYDEGGER, 60 EAST SOUTH TEMPLE, 1000 EAGLE GATE TOWER, SALT LAKE CITY, UT, 84111, US

(73) Assignee: ALPHAMED MEDIZINTECHNIK FISCHER GMBH, Lassnitzhohe (AT)

(21) Appl. No.: 11/718,789

(22) PCT Filed: Nov. 8, 2005
(86) PCT No.: PCT/AT05/00443

§ 371 Date: May 17, 2007

(30) Foreign Application Priority Data
Nov. 8, 2004 (AT) A 1862/2004

(51) Int. Cl. A61F 2/42

(52) U.S. Cl. 623/21.18

(57) ABSTRACT

The invention relates to a talus component for a multipart ankle joint prosthesis, which talus component has a bottom side facing the osseous talus after implantation, and is characterized in that the bottom side facing the osseous talus is configured as a concave spherical segment.

The present invention relates to ankle joint prosthesis elements, more specifically a talus component and a tibia component-inlay combination for a multipart ankle joint prosthesis, as well as ankle joint prostheses composed of said elements.

In view of the numerous reasons which argue for a flexible solution for a painfully destructed upper ankle joint, it has been only logical after the first successes in hip and knee joint endoprosthetics to search for appropriate solutions also for other joints, particularly since no promising alternative in terms of a resection arthroplasty has been available for joints directly burdened by the body weight.

The earliest ankle joint prostheses were cemented metal-polyethylene joints. The best known prosthesis of this generation, the so-called St. Georg endoprosthesis, exhibited a slide-like talus component which articulated with a tibia component shaped downwards in a congruent, i.e., concave manner.

The second generation of endoprostheses, which were now implantable also in a cement-free fashion, was based on the concept of “meniscal” Oxford knee endoprostheses. The slide-like shape of the metallic talus component was maintained by means of a superficial guide for a polyethylene slide core shaped congruently and articulating upwards in a freely movable manner with the planar bottom surface of the tibia component. In this way, the slide core (inlay) is secured against luxations due to the bottom surface which, accordingly, is shaped congruently toward the talus trochlea, however, upwardly, it still allows all directions of movement, among other things, in the rotational sense and anterodorsally.

In principle, it has become apparent that only an ankle joint prosthesis having an anatomically and biomechanically compatible design which is implanted with a minimum amount of bone resection and is guided in a manner that is free from the remaining ligaments and tendons can meet the mechanical demands and thus comply with a high long-term survival rate. In consideration of these requirements, various types of ankle joint prostheses have been developed and used for treatment.

The endoprostheses used today can basically be divided into mono- and multiaxial two-component endoprostheses as well as three-component endoprostheses. Three-component endoprostheses are generally composed of a tibia component and a talus component as well as an intermediate slide core.

A three-component prosthesis (HINTEGRA®; Newdeal SA, Vienne, France) comprising anatomical tibia and talus components as well as a freely movable polyethylene (PE) slide core of ultra-high density (inlay) is known, for example. The tibia component comprises a metal plate with small anchoring pyramids on the tibial side as well as a ventral shield which permits screw fixation through two oval holes and prevents the ingrowth of scar tissue and hence limited movement. The talus component has a shape which is similar to a cone with a narrower medial lateral radius. A medial and lateral rim guides the PE slide core. Said mobile and guided slide core has a flat surface toward the tibia component and a concave surface toward the talus component.

In another known ankle joint prosthesis (“A.E.S-Prothese”, Biomet Merck Deutschland GmbH), the tibia component consists of a metal plate comprising an intraoperatively attachable keel whereas the talus component is configured as part of a cylinder surface which, in the circumferential direction, has a central groove-shaped incision in which the inlay, which is shaped correspondingly on the talus side, slides. The top side of the inlay has a planar design corresponding to the tibia component.

The tibia component of a further known prosthesis (LINK S.T.A.R.®; Waldemar Link GmbH & Co. KG) consists of a metal plate having a mirror-finished flat articulation surface which has two cylindrical fixation bars arranged on the backside for anchoring in the tibia bone. A convex metal cap which, on its bottom side, converges in a rounded obtuse angle serves as the talar component. A rib for guiding the PE inlay which, accordingly, has a concave surface with a groove is located in the centre of the convex surface of the talus component, running in an A/P direction.

It is an object of the present invention to provide ankle joint prosthesis elements and ankle joint prostheses, respectively, which show an optimum fit of the talar prosthesis portion despite little bone resection. In addition, a lateral protrusion of the slide core and hence the development of impingement are to be avoided.

With a talus component for a multipart ankle joint prosthesis, which talus component has a bottom side facing the osseous talus after implantation, said object is achieved according to the invention in that the bottom side facing the osseous talus is configured as a concave spherical segment.

It has turned out that the concave design of the bottom side of the talus component allows a particularly gentle preparation of the bone.

In a preferred embodiment, a means for anchoring in the bone is provided on the bottom side configured as a concave spherical segment.

Preferably, the means comprises a centrally arranged tubular shaft and a pin arranged ventrally thereto.

An object of the invention is also a three-part ankle joint prosthesis comprising a talus component, an inlay and a tibia component, which is characterized in that a talus component according to the invention is provided.

A further aspect of the invention relates to a tibia component-inlay combination for a multipart ankle joint prosthesis, wherein the tibia component comprises a plate and a means for anchoring in the bone and the inlay has a planar tibial surface. According to the invention, a substantially central elevation is provided on the side of the plate facing the inlay and a recess for the elevation is provided on the tibial surface of the inlay, with the recess being designed such that the inlay is allowed to rotate and slide in an AP-direction relative to the tibia component and a lateral movement of the inlay is prevented.

In this manner, a lateral protrusion of the slide core and hence the development of impingement are avoided.

According to a preferred embodiment, the elevation is designed in the shape of a spherical segment and the recess is configured as a segment of a cylinder having spherical-segment-shaped ends, with the axis of the cylinder being oriented in the A/P-direction.

In the above-described embodiment, the recess is provided in the inlay and the elevation engaging said recess is provided in the plate of the tibia component. According to a further preferred embodiment, it is just the opposite: Said embodiment therefore concerns a tibia component-inlay combination for a multipart ankle joint prosthesis, wherein the tibia component comprises a plate and a means for anchoring in the bone and the inlay has a planar tibial surface, with a substantially central recess being provided on the side of the plate facing the inlay and an elevation for said recess being provided on the tibial surface of the inlay. The recess is designed such that the inlay is allowed to rotate and slide in an AP-direction relative to the tibia component and a lateral movement of the inlay is prevented.

Preferably, the elevation is designed in the shape of a spherical segment and the recess is configured as a segment of a cylinder having spherical-segment-shaped ends, with the axis of the cylinder being oriented in the A/P-direction.

The means for anchoring in the bone preferably comprises a ventrally curved keel.

Further objects of the invention relate to three-part ankle joint prostheses which comprise the tibia component-inlay combination according to the invention or both the talus component according to the invention and the tibia component-inlay combination.

Below, the invention is illustrated in further detail on the basis of the drawing, wherein

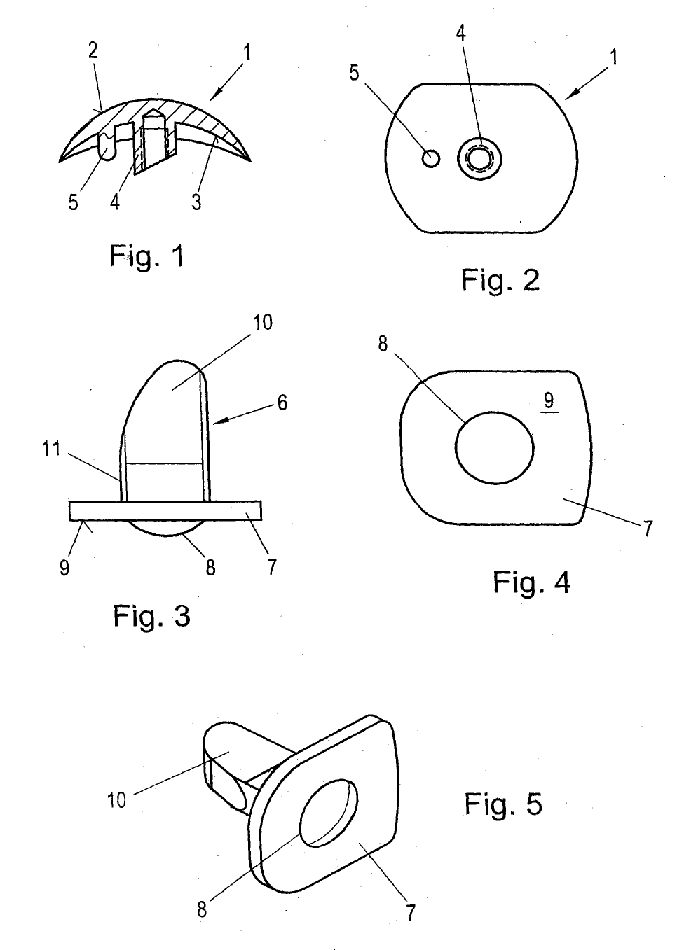

FIGS. 1 and 2 show a cross-section and a view, respectively, of an embodiment of the talus component according to the invention,

FIGS. 3, 4 and 5 show views of an embodiment of the tibia component of a tibia component-inlay combination according to the invention and

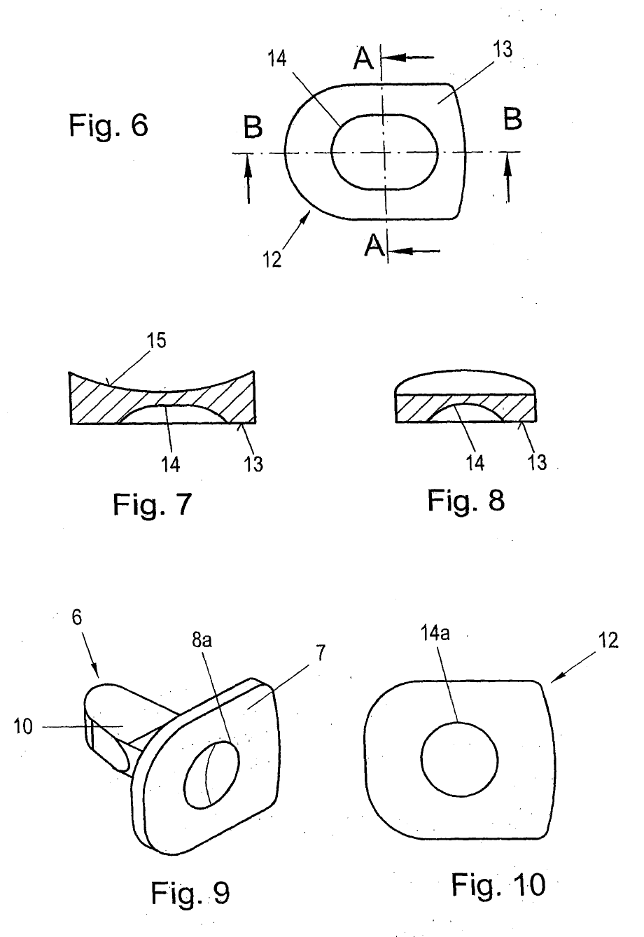

FIGS. 6, 7 and 8 show a view and cross-sections, respectively, of an embodiment of the inlay of a tibia component-inlay combination according to the invention.

FIGS. 9 and 10 show a further embodiment of a tibia component and of an inlay, respectively.

FIG. 1 shows a section through an embodiment of the talus component 1 according to the invention. Said element is usually manufactured from a biocompatible metal, for example, from CoCrMo according to ISO 5832/4. Reference numeral 2 denotes the cylindrically convex surface facing the inlay. The element's bottom side which is configured as a concave spherical segment, faces the talus and can be coated, e.g., with tricalcium phosphate is indicated by 3.

FIG. 2 shows a view of the bottom side 3 of the talus component 1. In the central part of the bottom side 3, a tubular shaft 4 is arranged which serves for the anchorage in the bone and, as illustrated, can be provided with a female thread. Said shaft is oriented in the direction of the central radial of the spherical surface. In order to facilitate the insertion of the articular part, the tubular shaft 4 is chamfered such that the longer portion is located at the front.

A metal pin 5 is moulded ventrally to the tubular shaft 4. Said pin has, for example, a diameter of about 3 mm and, in the illustration, is arranged in parallel to the direction of the central shaft 4. The pin 5 serves as the primary safeguard against rotation of the inserted talar prosthesis element.

In FIG. 3, a side view of an embodiment of the tibia component 6 of a tibia component-inlay combination according to the invention is illustrated. The tibial prosthesis element consists of a metal plate 7 having a thickness of, e.g, 3 mm, which, on the inlay's side, exhibits a spherical-segment-shaped elevation 8 forming a part for the necessary restricted guidance of the polyinlay. The elevation 8 is provided essentially in a central position on the bottom side 9 of the metal plate 7, as shown in FIGS. 4 and 5.

For enlarging the osseous integration area and for safeguarding against rotation of the tibia component 6, said component is provided with a keel 10 on the cranial side. Said keel 10 is clearly flattened on its lower boundary face 11 in order to facilitate the insertion of the tibia component 6. The cranial keel portion tapers continuously and is thus supposed to contribute to the impaction of the spongy bone.

Preferably, keels of various lengths can be individually attached onto the respective metal plate 7, according to requirements. Thereby, the longest revision keel preferably has 2 holes for the reception of lock pins. A fastening mechanism for modular tibia keels can be integrated in the spherical-segment-shaped elevation 8 as well as in the metal plate 7.

FIG. 6 shows a view of the tibial surface 13 of the PE inlay 12, which can be manufactured, for example, from Chirulen ISO 5834. The configuration of the inlay 12 conforms, in terms of its shape, with the metal plate 7 of the tibia component 6. In principle, the size to be used for the inlay 12 is adjusted to the talus component.

The tibial surface 13 consists of a planar surface in the centre of which, in the longitudinal direction, i.e., in the A/R-direction, a recess 14 shaped like a groove is provided, which is designed as a cylindrical segment having spherical-segment-shaped ends. Said shape of the recess 14 can be seen from the cross-sections illustrated in FIGS. 7 and 8 and taken along line B-B and line A-A, respectively, of FIG. 6. The recess 14 receives the elevation 8 of the tibia component 6 in a closed manner. Upon interaction, the necessary sliding in an A/P-direction and the rotation of the inlay 12 relative to the tibia component 6 are thus ensured. However, the lateral movability relative to the tibia component 6 is restricted. With respect to the metal plate 7 of the tibia component 6, this prevents a lateral protrusion and hence the development of impingement by the inlay 12.

As illustrated in FIG. 7, the surface 15 of the inlay 12 facing the talus component 1 is configured, in accordance with the latter, as a concave cylinder surface.

In FIGS. 9 and 10, the reverse embodiment is shown in which the recess is not provided in the inlay but in the tibia component. In FIG. 9, the recess with reference numeral 8a is shown to be located in the plate 7 of the tibia component. The counterpart, i.e., the elevation, engaging the recess is then provided in the inlay 12, as indicated by reference numeral 14a in FIG. 10.

The recess 8a in the plate 7 can also be designed in the shape of a groove, analogous to the groove-shaped recess 14 of inlay 12 (FIG. 6), and also in the longitudinal direction, i.e., in the A/R-direction. In this case, the recess 8a is configured as a cylindrical segment having spherical-segment-shaped ends.

1. A talus component for a multipart ankle joint prosthesis, the talus component comprising a bottom side facing the osseous talus after implantation, wherein
the bottom side facing the osseous talus is configured as a concave spherical segment.

2. The talus component according to claim 1, wherein a means for anchoring in the bone is provided on the bottom side configured as a concave spherical segment.

3. The talus component according to claim 2, wherein the means comprises a centrally arranged tubular shaft and a pin arranged ventrally thereto.

4. A three-part ankle joint prosthesis comprising a talus component, an inlay and a tibia component wherein a talus component according to claim 1 is provided.

5. A tibia component-inlay combination for a multipart ankle joint prosthesis, wherein the tibia component comprises a plate and a means for anchoring in the bone and the inlay has a planar tibial surface, wherein
a substantially central elevation is provided on the side of the plate facing the inlay and a recess for the elevation is provided on the tibial surface of the inlay, with the recess being designed such that the inlay is allowed to rotate and slide in an AP-direction relative to the tibia component and a lateral movement of the inlay is prevented.

6. The tibia component-inlay combination according to claim 5, wherein the elevation is designed in the shape of a spherical segment and the recess is configured as a segment of a cylinder having spherical-segment-shaped ends, with the axis of the cylinder being oriented in the A/P-direction.

7. A tibia component-inlay combination for a multipart ankle joint prosthesis, wherein the tibia component comprises a plate and a means for anchoring in the bone and the inlay has a planar tibial surface, wherein
a substantially central recess is provided on the side of the plate facing the inlay and an elevation for said recess is provided on the tibial surface of the inlay, with the recess being designed such that the inlay is allowed to rotate and slide in an AP-direction relative to the tibia component and a lateral movement of the inlay is prevented.

8. The tibia component-inlay combination according to claim 7, wherein the elevation is designed in the shape of a spherical segment and the recess is configured as a segment of a cylinder having spherical-segment-shaped ends, with the axis of the cylinder being oriented in the A/P-direction.

9. The tibia component-inlay combination according to claim 5 wherein the means for anchoring in the bone comprises a ventrally curved keel.

10. A three-part ankle joint prosthesis comprising a talus component, an inlay and a tibia component, wherein, as the tibia component and the inlay, a combination according to claim 5 is provided.

11. A three-part ankle joint prosthesis comprising a talus component, an inlay and a tibia component, wherein a talus component comprising a bottom side facing the osseous talus after implantation is provided, wherein the bottom side facing the osseous talus is configured as a concave spherical segment and, as the tibia component and the inlay, a combination according to claim 5 is provided.

12. A three-part ankle joint prosthesis comprising a talus component, an inlay and a tibia component, wherein a talus component according to claim 2 is provided.

13. A three-part ankle joint prosthesis comprising a talus component, an inlay and a tibia component, wherein a talus component according to claim 3 is provided.

14. The tibia component-inlay combination according to claim 6, wherein the means for anchoring in the bone comprises a ventrally curved keel.

15. The tibia component-inlay combination according to claim 7, wherein the means for anchoring in the bone comprises a ventrally curved keel.

16. The tibia component-inlay combination according to claim 8, wherein the means for anchoring in the bone comprises a ventrally curved keel.

17. A three-part ankle joint prosthesis comprising a talus component, an inlay and a tibia component, wherein, as the tibia component and the inlay, a combination according to claim 6 is provided.

18. A three-part ankle joint prosthesis comprising a talus component, an inlay and a tibia component, wherein, as the tibia component and the inlay, a combination according to claim 7 is provided.

19. A three-part ankle joint prosthesis comprising a talus component, an inlay and a tibia component, wherein, as the tibia component and the inlay, a combination according to claim 8 is provided.

20. A three-part ankle joint prosthesis comprising a talus component, an inlay and a tibia component, wherein, as the tibia component and the inlay, a combination according to claim 9 is provided.

21. A three-part ankle joint prosthesis comprising a talus component, an inlay and a tibia component, wherein a talus component comprising a bottom side facing the osseous talus after implantation is provided, wherein the bottom side facing the osseous talus is configured as a concave spherical segment and, as the tibia component and the inlay, a combination according to claim 6 is provided.

22. A three-part ankle joint prosthesis comprising a talus component, an inlay and a tibia component, wherein a talus component comprising a bottom side facing the osseous talus after implantation is provided, wherein the bottom side facing the osseous talus is configured as a concave spherical segment and, as the tibia component and the inlay, a combination according to claim 7 is provided.

23. A three-part ankle joint prosthesis comprising a talus component, an inlay and a tibia component, wherein a talus component comprising a bottom side facing the osseous talus after implantation is provided, wherein the bottom side facing the osseous talus is configured as a concave spherical segment and, as the tibia component and the inlay, a combination according to claim 8 is provided.

24. A three-part ankle joint prosthesis comprising a talus component, an inlay and a tibia component, wherein a talus component comprising a bottom side facing the osseous talus after implantation is provided, wherein the bottom side facing the osseous talus is configured as a concave spherical segment and, as the tibia component and the inlay, a combination according to claim 9 is provided.

25. The talus component according to claim 11, wherein a means for anchoring in the bone is provided on the bottom side configured as a concave spherical segment.

26. The talus component according to claim 25, wherein the means comprises a centrally arranged tubular shaft and a pin arranged ventrally thereto.
